(19) United States
(12) Patent Application Publication
(10) Pub. No.: US 2011/0072564 A1
(43) Pub. Date: Mar. 31, 2011

(54) Sports Goggles with a Sports Helmet

(75) Inventor: Markus Krauter, Rudersberg (DE)

(73) Assignee: PROTEQ EUROPE S.C.S, Satu Mare (RO)

(21) Appl. No.: 12/894,201

(22) Filed: Sep. 30, 2010

(30) Foreign Application Priority Data
Sep. 30, 2009 (DE) 10 2009 048 606.2
Jan. 29, 2010 (DE) 10 2010 006 772.5

(51) Int. Cl. A42B 1/24; A42B 1/08; A61F 9/02

(52) U.S. Cl. 2/422; 2/424; 2/452

(57) ABSTRACT

Sports goggles for use with a sports helmet have a frameless lens or a frame for receiving a lens or several lens segments or lenses. At least one flexible and elastic fastening strap is connected to each side of the frameless lens or the frame, respectively. The at least one flexible and elastic fastening strap is detachably connected to a fastening device arranged in a lateral area of a shell of the sports helmet, respectively. The fastening device and the at least one fastening element are embodied as paired permanent magnets, respectively.

### BACKGROUND OF THE INVENTION

The invention concerns sports goggles with a sports helmet.

In many sports, for example, bicycling, downhill skiing, ski jumping or snowboarding, sports helmets and safety goggles are worn for safety reasons.

Known sports goggles have either temple pieces or a wide elastic band for fixation on the head of the wearer or on the circumference of the helmet. Temple piece systems are not suited for use with a sports helmet because the temple pieces will cause uncomfortable pressure points underneath the helmet. Sports goggles with a wide elastic band for attachment are known from skiing. The disadvantage is that the elastic band can easily slide off the helmet. Moreover, only an axial adjustability of the length of the elastic band is possible.

For example, DE 203 06 048 proposes a unit of goggles and helmet wherein the shell of the helmet on both of its lateral rims has a securing means that is provided to secure the elastic connection between helmet and goggles at least in the upper position. A disadvantage of this solution is the cumbersome manipulation especially with gloves.

U.S. Pat. No. 4,918,753 A concerns glasses laterally connected by means of elastic straps that are laterally attached to the frame on a mushroom-shaped attachment member on the shell of the helmet. This solution is also difficult to manipulate especially with gloves. Moreover, the glasses are only insufficiently attached to the face of the wearer because of the wide strap.

The object of the invention is to propose sports goggles with a sports helmet that provide improved wearing comfort and can be produced inexpensively.

### SUMMARY OF THE INVENTION

The sports goggles for use with a sports helmet have a frame with a curved lens. In an alternative embodiment, the frame receives two planar or curved lenses.

In an alternative inexpensive embodiment, the frameless sports goggles have a monolithically designed lens that has a cutout for the nose and is aerodynamically shaped such that air flows are deflected across the surface of the lens in a substantially laminar flow and will not come into contact with the eyes of the user.

The sports goggles comprise fastening straps that are connected to fastening elements, which, in order to connect the sports goggles to a helmet, correspond to fastening means positioned in the shell of the helmet.

The fastening means and the fastening elements are designed as paired permanent magnets. A permanent magnet that is advantageously designed as a button or lever is connected to the fastening straps of the sports goggles. The second, preferably cup-shaped, permanent magnet is connected to the shell of the helmet. Preferably, this magnet is arranged in a groove or guide on or in the shell of the helmet so as to be moveable and securable so that the pretension of the fastening straps can be individually adjusted. The use of the permanent magnets facilitates by self-centering action upon approach of the two paired permanent magnets the adaptation of the sports goggles with the sports helmet.

In a preferred embodiment, the two ends of a fastening strap are connected to the goggles. In the loop that is formed thereby a fastening element comprising a permanent magnet is arranged. In the lateral area of the shell of the helmet fastening means, also in the form of permanent magnets, are arranged such that between them and the permanent magnets of the fastening elements an attractive force is acting. In this way, the user, when putting on the goggles, must only position the fastening elements in the vicinity of the fastening means of the shell of the helmet and the connection is realized automatically as a result of the magnetic forces.

Preferably, paired permanent magnets (fastening means in the shell of the helmet-fastening element on the fastening straps) have in the contact surface a mating projection-recess. With this arrangement the magnet pair upon contact will center itself automatically. Moreover, an excellent holding of the magnet connection is achieved because forces in the connection plane can be absorbed better. Preferably, the projection/recess in the permanent magnets has a circular cylindrical shape but can also have the shape of a circular truncated cone. Hence a form- and force-fit connection is provided.

In another, also preferred, embodiment, the sports helmet has on or in the shell of the helmet in the lateral areas, preferably in the temple area, one or several grooves or guides in which the fastening means are axially slidable and/or positive-lockingly and/or force-lockingly secured. In this way, the pretension of the fastening straps can be individually adjusted with simple means. Advantageously, the mechanically acting fastening means are embodied as a bayonet connection or as a clamping eccentric that enable clamping or locking in the groove or guide.

In a further preferred embodiment the fastening straps are connected by means of lens connectors in the lens. In this connection, the lens connectors have an area with which they are connected to the lens.

Preferably, the connection of lens connectors and lens is realized by means of a bayonet connection with appropriate parts provided in the lens and at the lens connectors.

In order for the bayonet connection not to release during use, the lens connectors extend away from the bayonet connection as much as possible parallel and at a spacing to the lens. As a result of the tension in lateral direction during use, the bayonet connection can not release because the lens connectors cannot rotate. This has moreover the advantage that the fastening straps are guided and damage of the straps by contacting the edge of the lens can thus be prevented. Moreover, the extension of the lens connectors acts as a lever and the lens is substantially uniformly pressed against the face of the wearer. For this purpose, the lens connectors are preferably attached in the upper and lower lateral areas of the lens. Preferably, the bayonet connection is joined when the extension is pointing to the center of the goggles and is secured by rotation. Alternatively, there is also the possibility to have the extensions point up or down.

Preferably, a lens connector is a tube of plastic material with a sufficient diameter for receiving the fastening straps. One end of the tube is angled at approximately 90 degrees wherein the concrete angle depends on the geometric design of the lens and has thereat the means for the bayonet connection. In the direction toward the bayonet closure, the diameter of the tube increases slightly. Inexpensively, for mounting only the straps must be pushed through the extension and then the end that exits from the area for the bayonet connection is to be secured in the simplest way by means of a knot.

The sports helmet according to the invention for use with sports goggles has at least two fastening means arranged laterally on the shell of the helmet for receiving the fastening elements wherein the fastening means and fastening elements are embodied as paired permanent magnets.

The fastening arrangement according to the invention for attachment of sports goggles on a sports helmet comprises of at least one elastic fastening strap having at its ends lens connectors connected thereto and a fastening element comprising a permanent magnet and several holding stays for receiving a fastening strap.

Preferably, the lens connectors have means for attachment in the lens by means of a bayonet connection.

Furthermore, the lens connectors have extensions pointing away from the contact point that in the state fastened to the lens are spaced from and substantially extend parallel to the surface of the lens.

The sports goggles have on each outer side of the frame or the lens at least one, preferably two flexible elastic fastening straps with which the goggles are secured on the helmet. Preferably, the fastening straps are in the form of a loop whose ends are arranged on the protective goggles and whose loop radius is connected to a fastening element that enables the positive-locking and/or force-locking connection with the helmet, preferably with the outer shell of the helmet.

Preferably, the fastening straps are of elastic round braid, round rubber braid, round rubber, elastic cord, or elastic yarn with polyester or polyamide fibers. Advantageously, the fastening straps are made from neoprene band, because neoprene does not absorb liquid.

The lens has at the side facing the face contact pads applied in a dot shape or strip shape or circumferentially extending rim strips of elastic material for support on the face that ensure comfortable wearing and prevent that the lens is coming directly into contact with the face of the user.

In an advantageous embodiment, the contact pads are arranged in the area of the nose, forehead, and cheeks. By intermediate spaces between the contact pads venting of the eye area of the user is enabled so that transpiration in the eye area can be prevented.

In an especially preferred embodiment the contact pads can be cut to size and have a one-sided self-adhesive layer. The contact pads are added when purchasing the goggles as additional material and, before use, the user can match the goggles to his facial contour and the individual application purpose.

In order to increase the wear comfort, the contact pads or the circumferentially extending rim strips are advantageously embodied to absorb sweat.

It is furthermore preferred that the lens is comprised of an impact resistant plastic material, preferably polycarbonate. In this way, the resistance is increased and the risk of dangerous splinter formation in case of falls, for example, when skiing or bicycling, is reduced.

The sports helmet has at least two fastening means that are preferably arranged in the area of the temples (between eye and ear) of the user for receiving the fastening straps of the sports goggles. The positive-locking and/or force-locking fastening means are preferably arranged as projections or recesses on the surface of the shell of the helmet on which the loop-shaped fastening straps are secured. Preferably, the fastening means are formed as mushroom-shaped projections that project slightly from the shell of the helmet.

In a preferred embodiment, the sports helmet has at both temple areas several spaced-apart fastening means. In this way, the position of the goggles (slant angle relative to the vertical) on the face of the user can be varied. Moreover, the length of the fastening straps can be compensated by guiding across several spaced-apart fastening means and in this way the contact pressure on the face of the user can be adjusted individually. Also, the sports goggles, as needed, can be used by a different user and with simple means can be matched to the ergonomic particularities.

In a further, also preferred, embodiment, the self-adhesive fastening means can be retrofitted by the user individually on the shell of the helmet.

In a preferred embodiment, each of the loop-shaped fastening straps has a tensioning element that is arranged slidably on the paired fastening straps. By sliding the tensioning elements in the direction towards the goggles, the segments of each fastening strap that converge away from the fastening means toward the goggles can be contracted and, in this way, the tension of the fastening straps can be increased. By moving the tensioning element in the direction of the fastening element, the tension of the fastening strap is reduced. In this way, the pressure of the goggles against the face of the wearer can be regulated.

In a further, also preferred, embodiment the fastening means has a clamping lever that is supported on a rotary axis of the fastening means so as to be rotatably or pivotably supported. The clamping lever has a bore or an open or closed passage that receives the loop-shaped fastening strap. By pivoting the clamping lever the pretension of the fastening strap is released and the sports goggles can be taken off easily. When putting on the sports goggles, by pivoting the clamping lever in the opposite direction, the required pretension is generated in order to enable a safe seat of the goggles on the face of the wearer. The fine adjustment and setting of the tension of the fastening straps are realized advantageously by the already described axially moveable tensioning element.

Because of the minimal number of parts and the constructively simple configuration, the sports goggles can be manufactured inexpensively. A further advantage of the sports goggles according to the invention resides in their stability because there is no temple piece system that can be damaged by pressure application. Further advantages are the extremely minimal weight and the possibility of individual adjustment of the position of the sports goggles in the area of the facial skull by means of the flexible loop-like fastening straps and the spaced-apart fastening means.

A specially advantage resides in that the sports goggles when being taken off (for example, when waiting at the ski lift) can remain attached by only one fastening means. In this position, the sports goggles cannot slide off the helmet (as is the case in known solutions of the prior art with a wide elastic band that surrounds the entire helmet). Since the sports goggles are in a suspended position in the temple area of the sports helmet, the field of view of the wearer is unobstructed.

Moreover, the sports goggles will not fog up by rising breath of the wearer. As a result of the preferably employed self-centering fastening means, the sports goggles can also be put on again easily and comfortably even with gloves.

### BRIEF DESCRIPTION OF THE DRAWINGS

In the following, several embodiments of the invention will be described and explained in connection with the drawings.

FIG. 1 shows sports goggles with a sports helmet with a tensioning element that is movable on the loop-shaped fastening straps and a pivot lever arranged on the fastening means.

FIG. 2 shows a side of an alternative embodiment of the fastening element that in the attached state is facing the helmet.

FIG. 3 shows sports goggles with a fastening element according to FIG. 2.

### DESCRIPTION OF PREFERRED EMBODIMENTS

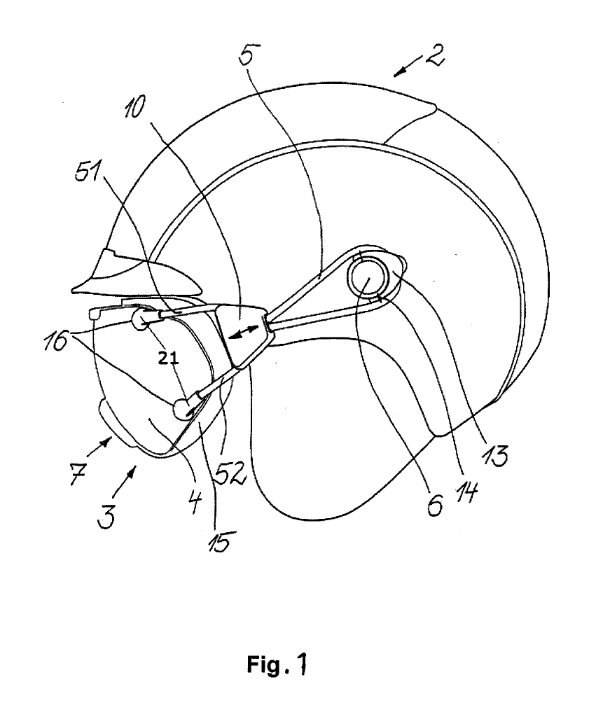

FIG. 1 shows sports goggles 3 with frame 15. On the frame 15 in the rim area on either side loop-shaped fastening straps 5 are connected by means of lens connectors 21 to the lens 4 at two contact points 16. The lens connectors 21 are connected by means of a bayonet connection in the lens. In this connection, on the upper strand 51 of the fastening strap 5 as well as on the lower strand 52 of the fastening strap 5 one lens connector 21 is arranged, respectively. The lens connectors 21 at the end of the two upper strands 51 of the fastening straps 5 are attached in the upper lateral area of the lens and the lens connectors 21 at the ends of the two lower strands 52 of the fastening straps 5 are correspondingly connected in the lower lateral area of the lens 4. In this way, the unobstructed view of the user is not limited by the lens connectors 21.

The lens connectors 21, as described above, are connected by means of a bayonet connection in the lens 4 wherein said connectors 21 are inserted while oriented toward the center of the lens 4 and then, by rotation in the direction of the outer side of the lens 4, are fastened within the lens 4. The lens connectors 21 are thus rotatable upon use and are thus adaptable to the face and the shape of the head of the user. Away from the attachment within the lens 4, the lens connectors 21 extend away from the contact point 16 spaced approximately at a 2 cm spacing and substantially parallel to the lens.

The other loop-shaped end of the fastening strap 5 is connected to a clamping lever 13 that is arranged pivotably on the fastening element 6.

The clamping lever 13 has a closed channel which receives the loop-shaped fastening strap 5. By pivoting the clamping lever 13 out of the illustrated position in the direction of the sports goggles 3 the pretension of the fastening strap 5 is released and the sports goggles 3 can be easily taken off. When putting on the sports goggles 3, the required pretension is generated by pivoting the clamping lever 13 in the opposite direction in order to enable a safe seat of the goggles on the face of the wearer 1. Fine adjustment and setting of the tension of the fastening straps 5 is realized by the already axially movable tensioning element 10. The tensioning element 10 is embodied as an injection-molded plastic part. In the trapezoid housing of the tensioning element there are four deflection pins arranged about which the upper and lower strands 51, 52 of the fastening strap 5 are guided.

The fastener 6 is embodied of a fastening element on the goggles and fastening means on the helmet in the form of paired permanent magnets. The outer pin-shaped part of the fastener 6 projects positive-lockingly and force-lockingly into the lower cup-shaped permanent magnet, not illustrated in detail. With a conical design of the fastener 6 the closure has a self-centering action.

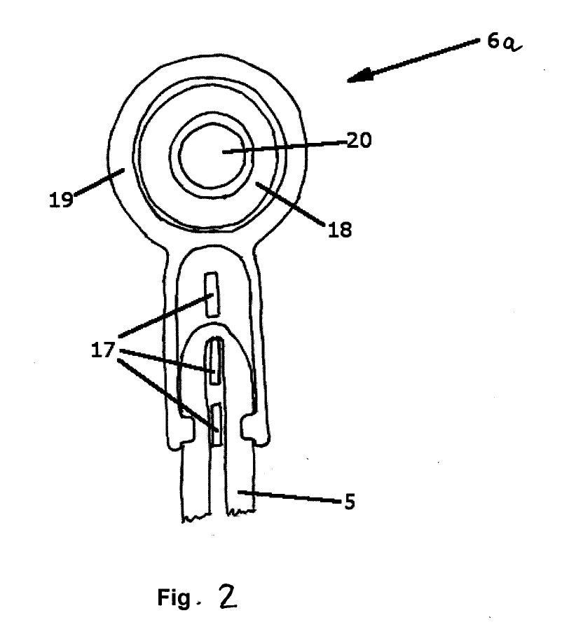

FIG. 2 shows the side of an alternative embodiment of a fastening element 6a that is facing the helmet in the fastening state and is of an especially simple configuration. The fastening element 6a has three holding stays 17 for the fastening straps 5 with which the user can vary the length of the straps 5 and therefore can adjust them to his needs. The attachment on the helmet is realized, as described above, by means of a permanent magnet 18 that is movable to a limited extent within a frame 19. The permanent magnet 18 has centrally a projection 20 in the form of a circular cylinder stub. On the helmet, not shown in FIG. 2, there is also a permanent magnet as a fastening means arranged in the shell of the helmet as a counter member. By means of the configuration of the permanent magnets 18 with the central projection 20 a safe attachment is ensured because the projection/recess counteracts pull in the direction of the shell of the helmet. On the other hand, the position of the two magnets relative to one another is automatically self-centered during attachment so that a precise and safe holding of the goggles is ensured. The goggles can thus be easily attached even with gloves in that the fastening means must only be moved into the vicinity of the permanent magnets arranged in the shell of the helmet where they will automatically connect and center themselves.

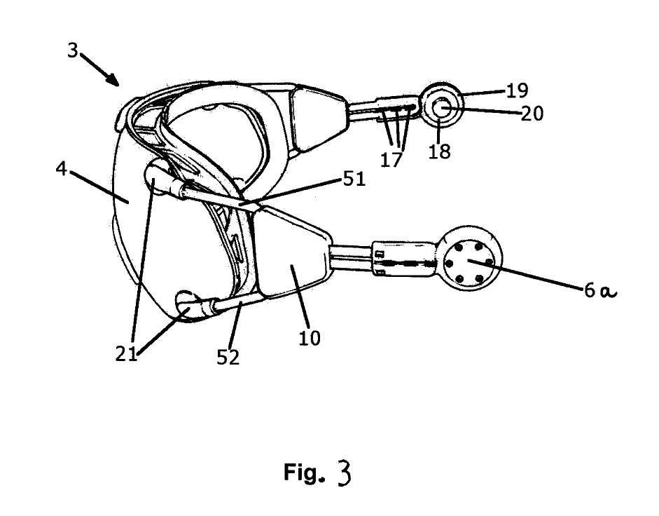

FIG. 3 shows goggles 3 with a fastening element 6a in accordance with the alternative embodiment according to FIG. 2 with a tensioning element 10 and the lens connectors 21. The lens connectors 21 are arranged in such a way on the rim of the lens 4 that the extensions project past the edge of the lens. In this way, the fastening strands 51, 52 will not become damaged by rubbing on the edge of the lens 4. By means of the tensioning element 10, the user can regulate the pressing force of the goggles 3 on the face. By means of the adjusting possibility of the length of the fastening straps 5 by means of the securing stays 17 in the fastening element 6 (FIG. 2) in connection with the tensioning element 10, the goggles provide excellent adjustability for almost any face.

The specification incorporates by reference the entire disclosure of German priority documents 10 2009 048 606.2 having a filing date of Sep. 30, 2009 and 10 2010 006 772.5 having a filing date of Jan. 29, 2010.

While specific embodiments of the invention have been shown and described in detail to illustrate the inventive principles, it will be understood that the invention may be embodied otherwise without departing from such principles.

### LIST OF REFERENCE NUMERALS

2 sports helmet
3 sports goggles
4 lens
5 fastening strap
51 upper strand of the fastening strap 5
52 lower strand of the fastening strap 5
6 fastener
6a fastening element
7 nose cutout
10 tensioning element
13 clamping lever
14 axis of rotation of the pivot lever
15 frame of the sports goggles
16 contact point
17 holding stay
18 permanent magnet
19 frame
20 projection
21 lens connector

What is claimed is:

1. Sports goggles for use with a sports helmet, comprising:
a frameless lens or a frame for receiving a lens or several lens segments or lenses;
at least one flexible and elastic fastening strap connected to each side of the frameless lens or the frame, respectively;
wherein the at least one flexible and elastic fastening strap has a fastening element that is detachably connected to a fastening means arranged in a lateral area of a shell of the sports helmet, respectively;
wherein the fastening means and the fastening element are embodied as paired permanent magnets, respectively.

2. The sports goggles according to claim 1, wherein the at least one flexible and elastic fastening strap is force-lockingly and/or positive-lockingly connected to the fastening means.

3. The sports goggles according to claim 1, wherein the fastening means is arranged in a temple area of the shell of the sports helmet.

4. The sports goggles according to claim 1, wherein the at least one fastening strap is made of elastic round braid, round rubber braid, round rubber, elastic cord, or elastic yarn with polyester or polyamide fibers, or neoprene band.

5. The sports goggles according to claim 1, wherein two of the at least one flexible and elastic fastening strap are arranged on each side so as to be spaced apart, the sports goggles further comprising a frame-shaped or sleeve-shaped tensioning element surrounding the two spaced-apart fastening straps, wherein the tensioning element is arranged on the two spaced-apart fastening straps so as to be slidable in axial direction of the two spaced-apart fastening straps.

6. The sports goggles according to claim 1, wherein the at least one flexible and elastic fastening strap is attached by a lens connector in the lens.

7. The sports goggles according to claim 1, wherein the lens connector is embodied as a part of a bayonet connection with a matching part provided in the lens.

8. A sports helmet for use with sports goggles, wherein the sports helmet comprises a shell and at least two fastening means arranged laterally on the shell, wherein the fastening means are adapted to receive fastening elements of fastening straps of the sports goggles, wherein the fastening means and the fastening elements comprise permanent magnets.

9. A fastening arrangement for fastening sports goggles on a sports helmet, the fastening arrangement comprising at least one flexible and elastic fastening strap having ends provided with lens connectors and further comprising a fastening element, wherein the fastening element comprises a permanent magnet and several securing stays for receiving the at least one fastening strap.

10. The fastening arrangement according to claim 9, wherein the lens connectors are embodied as a part of a bayonet connection to be received in a matching part of the bayonet connection within the lens.

11. The fastening arrangement according to claim 9, wherein the lens connectors, when fastened within the lens at a contact point, extend away from the contact point at a spacing and substantially parallel to a surface of the lens.
